(19) United States
(12) Patent Application Publication
(10) Pub. No.: US 2001/0023695 A1
(43) Pub. Date: Sep. 27, 2001

(54) Nasal dilator

(76) Inventor: Thomas Auriemma, Jackson, NJ (US)

Correspondence Address: CLIFFORD G. FRAYNE, Suite 7A, 136 Drum Point Road, Brick, NJ, 08723, US

(21) Appl. No.: 09/779,575

(22) Filed: Feb. 9, 2001

Related U.S. Application Data
(60) Provisional application No. 60/185,470, filed on Feb. 28, 2000.

(51) Int. Cl. A61M 29/00; A62B 7/00; A62B 9/00; A61M 15/00; A61M 16/00; A62B 18/00; A61F 5/08; A62B 7/10; A61G 10/00; A62B 23/02

(52) U.S. Cl. 128/200.24; 128/206.11; 606/199; 606/204.45

(57) ABSTRACT

An adhesive nasal dilator for affixing to the cheek area of a user for improving breathing and lessening of nasal passageway constriction, the nasal dilator comprising a substrate member having an adhesive layer formed on one side thereof, said adhesive layer positioned proximate the cheek bone of the user and extended rearwardly towards the ear where the second end of the adhesive layer is affixed to a skin portion of the user so as to open the nasal passageway.

### BACKGROUND OF THE INVENTION

1. Field of the Invention

The present invention relates to nasal dilators, and more particularly to a nasal dilator which cooperates with the cheek bone area of the face to prevent the nasal passage of the nose from constricting during breathing.

This application claims the benefit of a filing date of provisional application No. 60/185,470 “Nasal Dilator”, filed Feb. 28, 2000.

2. Description of the Prior Art

A large portion of the human population suffers some difficulty in breathing. This difficulty may arise as a result of a physiological malformation of the nasal passageway, or an allergic reaction to environmental surroundings. Regardless of the cause, nasal blockage or partial intermittent nasal blockage can lead to discomfort in breathing and irregularities and disturbances during sleep, such as snoring.

For a physiological malformation, the most common solution is surgery and with respect to allergic reactions which affect the nasal passageway, the most common solution is to identify and avoid the allergic reactant.

Other alternatives have been developed which can provide relief to individuals suffering from breathing difficulties. These alternatives are commonly referred to as nasal dilators and these nasal dilators consist of one of two types. The first type is a nasal dilator which is secured to the exterior of the individual's nose and attempts to spread or open the nasal passageway by means of a bandage or other similar article which is under tension and is affixed to the nose in an attempt to spread the nasal passageway.

Other nasal dilators are cooperative with the nose and actually provide for a tubular member to be inserted into the nasal passageway. All of the nasal dilators heretofore known are associated with or are secured to or about the nose of the individual. None of the nasal dilators heretofore known are cooperative with the cheek bone area, the zygomatic bone of the facial portion of the individual and cooperate to dilate the nasal passageway and the sinus cavities in order to improve breathing.

### OBJECTS OF THE INVENTION

It is an object of the present invention to provide for a novel nasal dilator which is cooperative with the cheek bone portion of the individual's face in order to improve breathing.

It is a further object of the present invention to provide for a novel nasal dilator which can be adjustable to adjust tension on the cheek bone portion of the facial area of the individual.

It is a still further object of the present invention to provide for a novel nasal dilator for use in sports activities which improves breathing and also limits reflective glare to the eyes by pigmenting the material of the nasal dilator.

### SUMMARY OF THE INVENTION

A nasal dilator which in one embodiment comprises a flexible substrate having an adhesive affixed to one side thereof, one end of the adhesive side being first affixed to the cheek bone area (zygomatic bone) of the face of the individual and the second end of the substrate being extended towards the ear of the individual and affixed to the skin of the individual thereby opening the nasal passage on that side of the face to which it is affixed. A second embodiment comprising a flexible, resilient strap which extends about the head of the individual, the ends of the strap having affixed thereto an adhesive engageable with the individual's face in the area of the cheek bone (zygomatic bone), the resilient, flexible strap applying tension to the adhesive portion to dilate the nasal cavity.

### BRIEF DESCRIPTION OF THE DRAWINGS

These and other objects of the present invention will become readily apparent to those skilled in the art from the following detailed descriptions of the preferred embodiments, particularly when taken in conjunction with the drawings in which:

FIG. 1 is a front view of a face illustrating the use of a first embodiment of the nasal dilator of the present invention;

FIG. 2 is a perspective view of the first embodiment of the nasal dilator;

FIG. 3 is a bottom view of the first embodiment of the nasal dilator;

FIG. 4 is a bottom view of a variation of the first embodiment of the nasal dilator;

FIG. 5 is a front view of a face illustrating the second embodiment of the nasal dilator of the present invention;

FIG. 6 is a side view of the second embodiment of the nasal dilator of the present invention; and

FIG. 7 is a perspective partial exploded view of a variation of the second embodiment of the nasal dilator of the present invention.

FIG. 8 is a perspective exploded view of a third embodiment of the nasal dilator of the present invention.

### DETAILED DESCRIPTION OF THE DRAWINGS

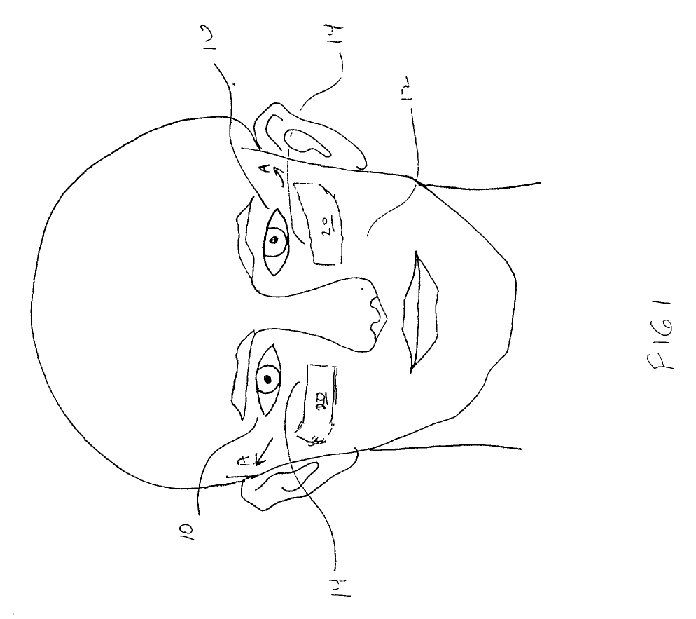

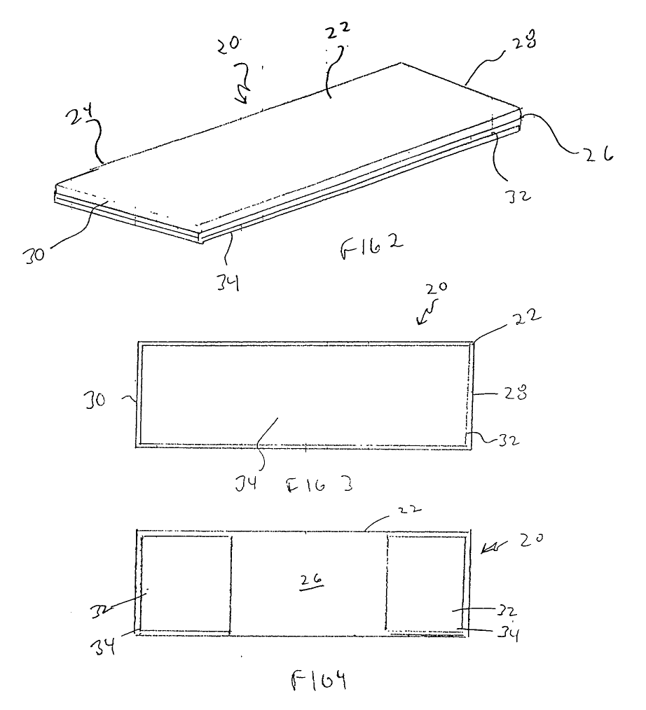

FIG. 1 is a front view of the face of an individual indicating the relative position between the eyes 10, the nose 12 and the zygomatic bone or hereinafter cheek bone 14. FIG. 1 illustrates the placement of a first embodiment of Applicant's nasal dilator 20 on the facial portion, approximate the cheek bone 14. The first embodiment of Applicant's nasal dilator 20, as illustrated in FIGS. 2 and 3, consists of a flexible resilient substrate 22 having a first side 24 and a second side 26 and ends 28 and 30. Second side 26 is overlaid with an adhesive layer 32 and adhesive layer 32 would be overlaid with a peelable removable layer 34.

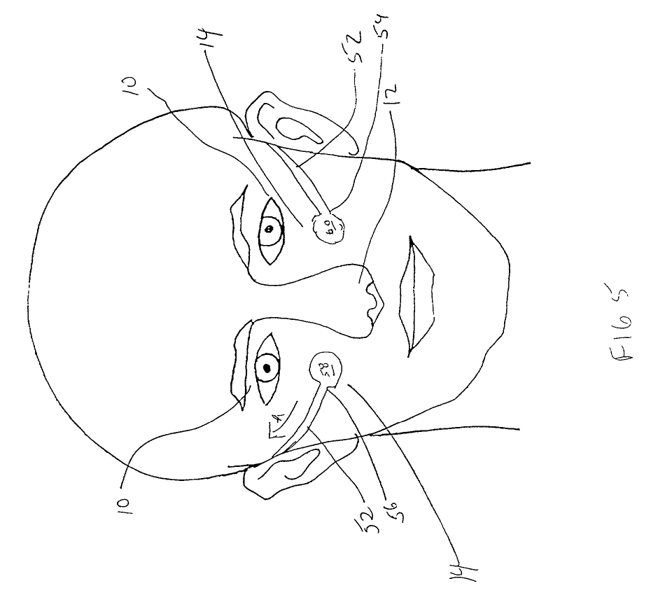

Nasal dilator 20 is comprised of a flexible resilient substrate 22 having a first side 24 and a second side 26, first side 24 being positioned outwardly from the skin. The second side 26 has applied thereto an adhesive layer 32 which can be coincident with the entire second side of nasal dilator substrate 22 or affixed to the end portions 28 and 30 as illustrated in FIG. 4. The adhesive layer 32 is overlaid with a peelable removable layer 34 to protect the adhesive layer 32 until it is time for application of the nasal dilator 20.

In application, the peelable, removable layer 34 is removed from the adhesive and end 28 of the nasal dilator is positioned on the face beneath the eye, adjacent the nose, and proximate the zygomatic bone, 10, 12, and 14 respectively. Substrate 22 is then drawn rearwardly in a direction designated by A, toward the ear whereupon second end 30 of the substrate 22 is affixed to the skin of the individual. In this embodiment, the nasal dilator 20 is affixed to the facial portion of the individual proximate the nose, beneath the eye and above the zygomatic or cheek bone and extends circumferentially rearwardly in the direction A towards the ear. This positioning serves to open the nasal passage and allow more ease in breathing.

If the individual were suffering from nasal distress in only one of the nasal passageways, the nasal dilator of the first embodiment could be applied only to that side of the face to which it was required. However, for best results in dilating the nasal passageways, an identical nasal dilator 20 would be applied in similar fashion to the opposite side of the nose of the individual.

Nasal dilators 20 could be worn during times of nasal distress in breathing such as when one is suffering from nasal congestion. The nasal dilator 20 of the present invention could also be worn while sleeping in order to open the nasal passageways. Still further, the nasal dilator 20 of the present invention could also be worn during sporting activities to enhance the individuals breathing and in such instances, the first side 24 of nasal dilator 20 could be manufactured with a non-reflective, non-glare pigment in which case the nasal dilator 20 would serve a dual purpose of aiding in the individual's breathing during the sporting activity and also serve the purpose of decreasing glare or reflection to the eyes in a manner similar to an athlete placing black grease below his eyes when participating in a sporting event.

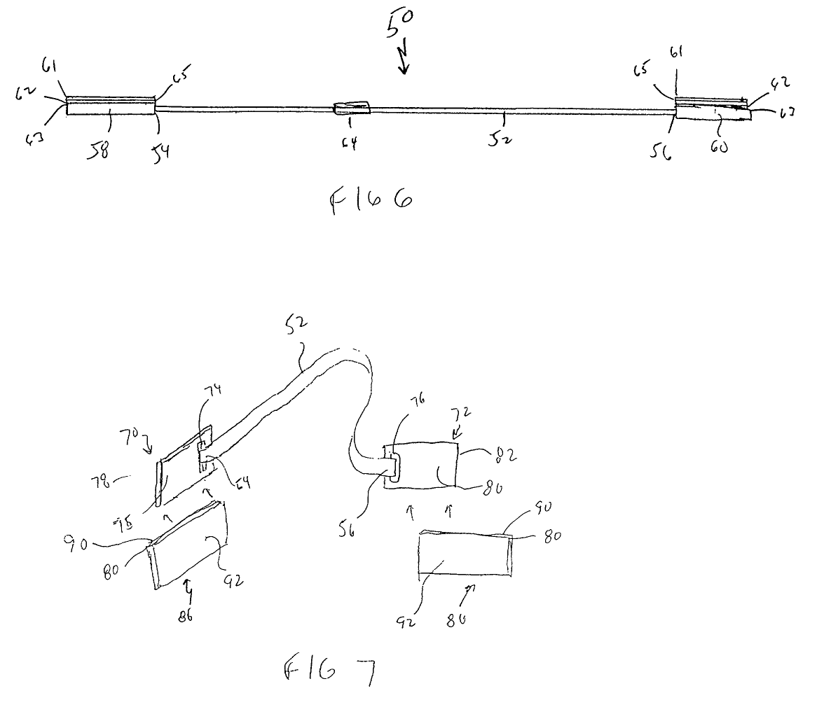

FIG. 5 is a front view of the face of an individual illustrating the positioning of a second embodiment of a nasal dilator generally referred to as 50. FIG. 6 is a side view of the second embodiment, nasal dilator 50.

The second embodiment, nasal dilator 50, is positioned on the facial portion of the individual proximate the nose 12, beneath the eye 10, and over the zygomatic or cheek bone 14. In this embodiment, the nasal dilator comprises a resilient, stretchable strap 52 designed to encircle the individual's head. Each end 54 and 56 of strap 52 enlarges to form a support member 58 and 60. Support members 58 and 60 are configured to receive a double-sided adhesive layer 62 one side of which 63 which would adhere to the support members 58 and 60. A removable protective layer 61 would be peelably removed to permit opposing side 65 to adhere to the facial portion proximate the zygomatic bone 14 of the individual. Such an adhesive layer 62 would be affixed to both support members 58 and 60 individually at ends 54 and 56 of strap 52. Additionally, strap 52 may have an adjustable tension means 64 to allow for the individual to adjust the tension of the double sided adhesive members 60 over the zygomatic bone in the direction A, towards the ears of the individual in order to dilate the nasal passageways.

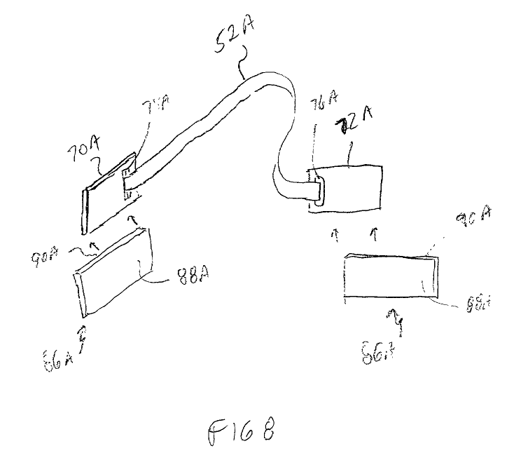

FIG. 7 is a perspective view of a variation of the second embodiment as illustrated in FIGS. 5 and 6. In this variation, the support members are not integrally formed with the strap member 52 as is the case with FIGS. 5 and 6. Strap 52 is secured at its ends 54 and 56 to semi-rigid support members 70 and 72 by means of a strap receiving aperture 74 and 76 respectively. Support members 70 and 72 are generally planar and defined by a first side 75 and a second side 78 with respect to support members 70 and first side 80 and second side 82 with respect to support member 72.

A disposable adhesive member 86 defined by a substrate layer 88 and adhesive layers 90 and 92 on opposing sides thereof is mated to support members 70 and 72 and then affixed to the cheek bone area 14 of the face with strap 52 extending about the individual's head. Adhesive member 86 would be disposable in this configuration whereas strap 52 and support member 70 and 72 would be reusable. The adhesive member 86 would be packaged with a peelable protective layer (not shown) similar to that illustrated in FIG. 6 which would be removable from one or both sides of the adhesive member 86 before the adhesive member was affixed to the support member on either side 75 or 78 of support member 70 or side 80 or 82 of support member 72.

In this configuration, the adhesive member 86 could be formed of the same general cross-sectional area as support member 70 or 72 or it could be of a greater cross-sectional area than support members 70 and 72 such that it would extend beyond the edges of the support member 70 and 72. The nasal dilator of the configuration as illustrated in FIG. 7 would be applied to the face in the same manner as the nasal dilator illustrated in FIGS. 5 and 6. The nasal dilator of FIG. 7 would be constructed of strap 52 and support members 70 and 72 which would permit their reusability whereas it is the intent that the nasal dilator of FIGS. 5 and 6 would be constructed of recyclable materials such that the entire nasal dilator could be discarded after use.

FIG. 8 illustrates the configuration where the adhesive members would be of greater cross-sectional area than the support members as illustrated in FIG. 7. In FIG. 8, strap 52A is again secured to semi-rigid support member 70A and 72A by means of strap receiving aperture 74A and 76A. In this instance, the disposable adhesive members 86A are defined by a substrate layer 88A and a single one-sided adhesive layer 90A, a portion of which is affixed to the semi-rigid support member 70A and 72A and a portion of the adhesive layer 90A extends beyond the periphery of the support member 70A and 72A for contact with the cheek bone area of the user.

While the present invention has been described with respect to the exemplary embodiments thereof, it will be recognized by those of ordinary skill in the art that many changes and amendments may be made without departing from the spirit and scope of the invention. Therefore it is manifestly intended that the scope of the invention be limited only by the claims and the equivalents thereof.

I claim:

1. An adhesive nasal dilator for affixing to the cheek area of the user for improved breathing and lessening of nasal passageway constriction, said nasal dilator comprising:
a flexible resilient substrate member having a first end and a second end and having a first side and a second side, said second side having an adhesive layer formed thereon, said first end of said substrate member having said adhesive layer positioned on a skin portion of the user proximate the cheek bone of said user, said second end of said substrate member having said adhesive layer formed thereon engaged under tension with a skin portion of the user proximate the ear of said user.

2. The adhesive nasal dilator in accordance with claim 1 wherein said adhesive layer is continuous on said second side of said substrate member.

3. The adhesive nasal dilator in accordance with claim 1 wherein said adhesive layer is positioned proximate said first end and said second end of said second side of said substrate member.

4. The adhesive nasal dilator in accordance with claim 1 wherein said first side of said flexible resilient substrate member is pigmented so as to be anti-glare and anti-reflective.

5. An adhesive dilator for affixing to the cheek area of a user for improving breathing and lessening of nasal passageway constriction, said nasal dilator comprising:
a pair of substrate members each having a first side and a second side, each of said substrate members having positioned on said second side, an adhesive member, said substrate members secured to each other by a flexible strap member, such that said substrate members and said adhesive member positioned thereon are adhesively positioned proximate the cheek bone of a user, said strap member extending around the head of the user, said strap member adjustable so as to place pulling tension to said substrate members toward the respective ears of the user

6. The adhesive nasal dilator in accordance with claim 5 wherein said substrate member and said adhesive member are unitarily formed and removably replaceable from said strap member.

7. The adhesive nasal dilator in accordance with claim 5 wherein said adhesive member is double layered and removably affixable to said substrate member.

8. The adhesive nasal dilator in accordance with claim 5 wherein said adhesive member is single layered and is removably attached to said substrate and a portion of which extends beyond the periphery of said substrate for contact proximate said cheek bone of said user.

9. The adhesive nasal dilator in accordance with claim 5 wherein said strap member is flexibly resilient.

10. The adhesive nasal dilator in accordance with claim 5 wherein said substrate member is pigmented so as to be anti-glare and anti-reflective.
